(19) United States
(12) Patent Application Publication
(10) Pub. No.: US 2024/0374495 A1
(43) Pub. Date: Nov. 14, 2024

(54) HUMAN MILK OLIGOSACCHARIDES IN CLEANSING COMPOSITION

(71) Applicant: DSM IP ASSETS B.V., Heerlen (NL)

(72) Inventors: Lise Anne KOHLER, Kaiseraugst (CH); Pascal Christian SIEBER, Kaiseraugst (CH)

(21) Appl. No.: 18/683,627

(22) PCT Filed: Aug. 23, 2022
(86) PCT No.: PCT/EP2022/073479

§ 371 Date: Feb. 14, 2024

(30) Foreign Application Priority Data
Aug. 24, 2021 (EP) 21192701.7

(51) Int. Cl. A61K 8/60; A61K 8/04; A61K 8/36; A61K 8/37; A61K 8/44; A61K 8/86; A61Q 19/10

(52) U.S. Cl. CPC A61K 8/60; A61K 8/046; A61K 8/361; A61K 8/375; A61K 8/442; A61K 8/602; A61K 8/86; A61Q 19/10

(57) ABSTRACT

The present invention relates to cleansing compositions comprising certain soaps and human milk oligosaccharides as well as to a method of increasing the foaming properties of cleansing compositions comprising at least one surfactant by incorporation of one or more human milk oligosaccharides into said cleansing composition.

The present invention relates to cleansing compositions comprising certain soaps and human milk oligosaccharides as well as to a method of increasing the foaming properties of cleansing compositions comprising at least one surfactant by incorporation of one or more human milk oligosaccharides into said cleansing composition.

In a number of cleansing products including shampoos, soaps, detergents, toothpastes and body washes consumers are looking for a high foaming capacity. For example, a shampoo that does not produce enough creamy, stable foam during shampooing has no chance of success on the market. The same applies to manual dishwashing detergents, even though a direct connection between the foaming capacity and the cleaning performance cannot be established at all in many cases.

Hence, besides performance requirements, such as cleaning performance and dermatological compatibility, the foaming behavior is a further important product feature. However, not all surfactant mixtures which perform satisfactorily and are economical in use show the required foaming behavior.

Thus, there is an ongoing need of ingredients which are able to increase the foaming properties of a cleansing composition without compromising the cleansing properties thereof.

Surprisingly it has now been found that human milk oligosaccharides (HMOs) are able to significantly increase the foaming characteristics of cleansing compositions such as in particular of cleansing compositions comprising at least one soap.

Cleansing compositions comprising at least one soap selected from the group consisting of sodium or potassium salts of aliphatic acids having from about 8 to 22 carbon atoms and one or more human milk oligosaccharide selected from the group consisting of 2′-fucosyllactose, 3-fucosyllactose, difucosyllactose, lacto-N-fucopentaose I, lacto-N-neotetraose and lacto-N-tetraose are still novel.

Thus, in a first aspect, the present invention relates to cleansing compositions comprising at least one soap selected from the group consisting of sodium or potassium salts of aliphatic acids having from about 8 to 22 carbon atoms and one or more human milk oligosaccharide selected from the group consisting of 2′-fucosyllactose, 3-fucosyllactose, difucosyllactose, lacto-N-fucopentaose I, lacto-N-neotetraose and lacto-N-tetraose as well as mixtures thereof.

In another embodiment, the present invention relates to the use of at least one human milk oligosaccharide as foam enhancing agent respectively foam booster, preferably to enhance foam quantity (height/volume), more in particular in cleansing compositions comprising at least one surfactant, such as most preferably at least one soap.

In a third aspect the present invention relates to a method of increasing the foaming properties of a cleansing composition comprising at least one surfactant, such as preferably at least one soap, said method comprising the step of adding at least one human milk oligosaccharide into said cleansing composition and optionally appreciating the effect.

In a further embodiment, the present invention relates to a method for improving the foaming properties of one or more surfactants, such as preferably of at least one soap in a cleansing composition, which method comprises combining at least one human milk oligosaccharide with said surfactant(s), such as preferably soap(s).

The term ‘cleansing composition’ as used herein refers to compositions suitable for cleansing. Such compositions are characterized by comprising at least one surfactant and encompass shampoos, body washes, toothpastes and the like.

The term ‘foam-enhancing agent’ respectively ‘foam booster’ as used herein refers to an agent which favorably influences the foam properties of a surfactant and/or a cleansing composition with regard to foaming ability, foam stability (foam remaining after certain period at rest), foam quantity (volume/height) (associated with good cleaning effect), creaminess of the foam (associated with conditioning effect), foam density, texture of the foam and/or foam speed (foam produced after a very short period of time. In particular the term ‘foam-enhancing agent’ respectively ‘foam booster’ is to be understood to enhance the foam quantity (foam volume/height) compared to a control, i.e. in the absence of human milk oligosaccharide(s). In particular, the foam quantity (height/volume) increase according to the present invention is greater than 5%, preferably greater than 10%, more preferably greater than 30% as measured according to the cylinder shake method compared to control, i.e. the respective composition not comprising the HMO(s).

The cylinder shake method is performed by preparing an aqueous composition comprising at least one surfactant with/without one or more human milk oligosaccharide(s) (also referred to as HMO(s)), placing the test composition in a cylinder and stoppering the cylinder, agitating the test composition by vigorously shaking the tube vertically for a set period of time (15 s), and immediately measure the height (volume) of the foam in the cylinder at the completion of the agitation step.

Increased foam height (volume) compared to control, i.e. the test sample without HMO(s) indicates increased foam power.

The term ‘human milk oligosaccharides’ (HMOs) refers to a family of structurally diverse unconjugated glycans that are highly abundant in and unique to human milk.

Originally, HMOs were proposed to be prebiotic “bifidus factors,” or human milk glycans found to promote growth in Bifidobacterial species of the gut and found uniquely in the stool of breast-fed infants compared to formula fed infants.

HMOs are composed of the five monosaccharides glucose (Glc), galactose (Gal), N-acetylglucosamine (GlcNAc), fucose (Fuc) and sialic acid (Sia), with N-acetylneuraminic acid (Neu5Ac) as the predominant if not only form of Sia. More than two hundred different HMOs have been identified so far. The most important ones are 2′-fucosyllactose (2′ FL), lacto-N-neotetraose (LNnT), 3-fucosyllactose (3FL), difucosyllactose (DFL), lacto-N-fucopentaose I (LNFP I), 3′sialyllactose sodium salt (3′SL), 6′sialyllactose sodium salt (6′SL), and lacto-N-tetraose (LNT).

HMOs can be isolated from breast milk or they can be produced chemically or biochemically. HMOs are available commercially from a variety of producers.

For the purpose of the present invention the source of the HMO is not essential. It is clear that HMOs from different sources can be used.

Particularly suitable HMO's according to the present invention are fucosylated HMO's such as in particular α1-2 respectively α1-3 fucosylated HMO's; as well as lacto-N-(neo)tetraoses as well as any mixtures thereof.

Particularly preferred fucosylated HMO's according to the present invention are 2′-fucosyllactose (CAS No: 41263-94-9), 3-fucosyllactose (CAS No: 41312-47-4), difucosyllactose (also known as lactodifucotetraose; CAS No: 20768-11-0) and lacto-N-fucopentaose I (CAS No: 7578-25-8).

Particularly preferred lacto-(neo)N-tetraoses according to the present invention are lacto-N-tetraose (CAS No: 14116-68-8) and lacto-N-neotetraose (CAS No: 13007-32-4), which are highly abundant neutral core HMO's in human milk.

In all embodiments according to the present invention the total amount of the at least one HMO used according to the present invention is preferably at least 0.1 wt.-%, more preferably at least 0.2 wt.-% most preferably at least 0.5 wt.-% and/or up to 10 wt.-%, more preferably up to 5 wt.-%, most preferably up to 1 wt.-%. Even more preferably, the amount is selected in the range from 0.01 to 10 wt.-%, more preferably in the range from 0.1 to 7.5 wt.-%, most preferably in the range from 0.2 to 5 wt.-%, based on the total weight of the cleansing composition. Further suitable ranges are from 0.05 to 2.5 wt.-%, from 0.1 to 2.5 wt.-%, from 0.25 to 2.5 wt.-% and from 0.5 to 2 wt.-%. Particularly preferred ranges according to the present invention are from 0.1 to 1 wt.-%, from 0.2 to 1 wt.-% as well as from 0.5 to 1 wt.-%, such as from 0.25 to 0.75 wt.-% or from 0.3 to 0.5 wt.-%.

It is well understood, in all embodiments of the present invention, that the HMO may not only serve as a foaming agent but may also excerpt further positive effects on the skin.

Preferably in all embodiments of the present invention the at least one HMO is selected from the group consisting of 2′-fucosyllactose, 3-fucosyllactose, difucosyllactose, lacto-N-fucopentaose I, lacto-N-neotetraose and lacto-N-tetraose as well as mixtures thereof, preferably from 2′-fucosyllactose, difucosyllactose, lacto-N-neotetraose and lacto-N-tetraose as well as mixtures thereof, most preferably from lacto-N-tetraose and a mixture of difucosyllactose and 2′-fucosyllactose.

The term ‘soap’ is used herein in its popular sense, i.e., it refers to the alkali metal or alkanol ammonium salts of aliphatic, alkane- or alkene monocarboxylic acids as well as mixtures thereof. Sodium, potassium, magnesium, mono-, di- and tri-ethanol ammonium cations, or combinations thereof, are particularly suitable for the purposes of this invention, preferably, however, sodium or potassium soaps are used.

Particularly preferred soaps for the purpose of the present invention are the well-known alkali metal salts such as in particular the sodium and/or potassium salts of natural or synthetic aliphatic (alkanoic or alkenoic) acids having from about 8 to 22 carbon atoms, preferably from about 8 to about 20 carbon atoms, most preferably from about 10 to about 18 carbon atoms. Even more preferred are the salts of the respective saturated (alkanoic acid) acids.

It is furthermore preferred that the soaps used according to the present invention such as in the cleansing composition according to the present invention comprise at least 85% of fatty acids having from 12 to 18 carbon atoms.

Particularly preferred soaps to be used according to the present invention such as in in the cleansing compositions of the present invention are the sodium and/or potassium salts of stearic, palmitic, lauric and/or myristic acid.

It is well understood that the soap can be used as such or can be formed ‘in situ’ such as in a cleansing composition by adding the respective acid and the respective base to said composition.

The amount of the at least one soap in cleansing compositions according to the present invention is preferably selected in the range from 3 to 95 wt.-%.

Preferably in all embodiments of the present invention a mixture of at least two different soaps is used, such as more preferably a mixture of two or more soaps selected from the group consisting of sodium and/or potassium salts of stearic, palmitic, lauric and/or myristic acid. Most preferred in all embodiments of the present invention is the use of a mixture of the sodium and/or potassium salts of stearic, lauric and myristic acid.

If the cleansing composition according to the present invention is an (extruded) soap bar, the amount of the at least one soap in said cleansing composition is preferably selected in the range from 30 to 95 wt.-% more preferably in the range from 40 to 80 wt.-%, most preferably in the range from 45 to 65 wt.-%, based on the total weight of the cleansing composition.

If the composition is a liquid soap, or a cream soap, or melt and pour soap bar the amount of at least one soap in said cleansing composition is preferably selected in the range from 3 to 50 wt.-%, more preferably in the range from 5 to 40 wt.-%, most preferably in the range from 7.5 to 35 wt.-%, based on the total weight of the cleansing composition. Further suitable ranges are from 10 to 40 wt.-%, from 10 to 35 wt.-%, from 10 to 20 wt.-%, from 10 to 15 wt.-%, from 15 to 40 wt.-%, from 15 to 35 wt.-% from 20 to 35 wt.-% and from 25 to 35 wt.-%.

In all embodiments of the present invention, the ratio (weight/weight) of the at least one soap with all the definitions and preferences as given herein to the at least one HMO with all the definitions and preferences as given herein is preferably selected in the range of 100:1 to 1:1, more preferably in the range of 75:1 to 5:1, most preferably in the range of 60:1 to 10:1, such as in the range of 55:1 to 12:1.

Particularly suitable non-ionic surfactants to be used for the purpose of the present invention encompass alkyl (poly) glucosides, i.e. compounds of the general formula CₙH₂ₙ₊₁O(C₆H₁₀O₅)ₓH, with x 1 to 4 such as preferably decyl glucoside and lauryl glucoside; polyethyleneglycol (PEG) based surfactants such as in particular mono- and di-esters of fatty acids with polyethylene glycol such as PEG-8 laurate, PEG-8 dilaurate, PEG-100 stearate and PEG-150 distearate as well as ethoxylated fatty acid glycerides (PEG-n glyceryl acylates) such as PEG-4 castor oil, PEG-120 glyceryl stearate, triolein PEG-6 esters, PEG-200 hydrogenated glyceryl palmate and PEG-7 glyceryl cocoate; (poly) glyceryl fatty acid esters such as glyceryl myristate, glyceryl stearate, glyceryl palmitate lactate; ethoxylated glycerides; glycol esters and derivatives, mono-esters of either ethylene or propylene glycol such as preferably glycol acylate or propylene glycol acylate; ethers comprising aliphatic (C₆-C₁₈) primary or secondary linear or branched chain acids, alcohols or phenols which possess no functional grouping other than the terminal OH group of the Polyoxyethylenated (POE) chain as well as ethoxylated alcohols and propoxylated POE ethers such as preferably PEG ethers, PPG ethers, propylene glycol alkyl POE-n ethers; alkanolamides such as preferably N-acyl derivatives of monoethanolamine (MEA) and diethanolamine (DEA), ethoxylated or not; such as preferably PEG-n acylamides, coco mono- or di-ethanolamide, palmamide MEA, acylamide DEA; sorbitan/sorbitol esters such as acetylated sorbitan ethoxylated or not, polysorbate-n, sorbitan sequiisostearate; alkyl carbohydrates esters or sucrose esters resulting from trans-esterification of sucrose with fatty acid methyl esters or triglycerides such as preferably alkylpolysaccharides; amine oxides such as preferably cocoamidopropyl amine oxide and lauramine oxide.

Particularly suitable zwitterionic surfactants (i.e. surfactants having at the same time a positive and a negative charge, also referred to as amphoteric surfactants) according to the present invention encompass secondary or tertiary aliphatic amine derivatives with an aliphatic chain, linear or branched, containing at least 8 to 22 carbon atoms and one anionic group selected from the group of carboxylate, sulfonatephosphate or phosphonate; acyl/dialkyl ethylenediamines such as preferably acylamphoacetate, disodium acylamphodipropionate, sodium acylamphohydroxypropylsulfonate, disodium acylamphodiacetate, sodium acylamphopropionate and wherein the acyl group represents either an alkyl or alkenyl group which can be mon- or polyunsaturated and contains from 5 to 29 carbon atoms; N-alkyl amino acids or imino diacids such as preferably aminopropyl alkylglutamide, alkylaminopropionic acid, sodium alkylimino propionate, alkyl glycinates and carboxyglycinates, sodium cocoglycinates; betaines such as preferably alkyl (C₈-C₂₀) betaines, alkyl amidopropyl betaines (cocamidopropyl betaines), alkyl (C₈-C₂₀) amidoalkyl (C₁-C₆) betaines, alkyl sulphobetaines and alkyl (C₅-C₂₀) amidoalkyl (C₁-C₆) sulphobetaines.

Suitable anionic surfactants according to the invention include, but are not limited to aliphatic sulfate, aliphatic sulfonate (e.g., C₈ to C₂₂ sulfonate or disulfonate), aromatic sulfonate (e.g., alkyl benzene sulfonate), alkyl sulfosuccinates, alkyl and acyl taurates, alkyl and acyl sarcosinates, sulfoacetates, alkyl phosphates, carboxylate and isethionates as well as mixtures thereof.

Particularly suitable anionic surfactants to be used for the purpose of the present invention are alkyl sulfates such as preferably sodium, triethanolamine or ammonium lauryl sulfates; alkyl ether sulfates (or Alkyl PEG-n sulfates) such as preferably sodium or ammonium lauryl ether sulfate, laureth sulfate, sodium C₂₋₁₅ pareth sulfate; alkyl amido ether sulfates; alkylaryl polyether sulfates; monoglycerides sulfates; acyl isethionate salts such as preferably sodium acylisethionate, sodium cocoyl isethionate; alkylaryl sulfonates salts such as preferably sodium alkylbenzene sulfonate and/or sodium dodecylbenzene sulfonate; alkyl sulfonates salts such as preferably sodium alkenyl sulfonate (sodium C₁₂-14 olefin sulfonate), sodium alkylglyceride sulfonate (sodium cocomonoglyceride olefin sulfonate), sodium alkylether sulfonate (sodium C12-15 pareth-15 sulfonate) and/or sodium lauryl sulfoacetate; (di) sodium sulfosuccinates such as preferably sodium dialkyl sulfosuccinate (dioctyl sodium sulfosuccinate), disodium alkyl PEG-n sulfosuccinate, disodium alkylamido PEG-n sulfosuccinate (disodium oleamido MEA-sulfosuccinate), disodium alkylsulfosuccinate; alkyl phosphates (mono-esters) such as preferably TEA monolauryl phosphate; PEG-n alkyl phosphates such as preferably DEA oleth-10 phosphate; di PEG-n alkyl phosphates (di-esters) such as preferably dilaureth-4 phosphate; phospholipids (tri-esters) such as preferably lecithin; carboxylic acids ester, such as preferably mono-ester of di- or tri-carboxylic acids such as lactylates (sodium acyllactylate, calcium stearoyl lactylate), laureth-6 citrate, dinonoxynol-9 citrate; ether carboxylic acids such as preferably sodium PEG-n alkyl carboxylates, sodium trideceth-13 carboxylate, nonoynol-8 carboxylic acid, alkyl C₆-C₂₄ ether carboxylates polyoxyalkylenated; acyl glutamates such as preferably di-TEA palmitoyl aspartate and sodium hydrogenated tallow glutamate: Acyl peptides with various amino acids side groups such as preferably palmitoyl hydrolysed milk protein, sodium cocoyl hydrolysed soy protein, TEA-cocoyl hydrolysed collagen or other acyl hydrolysed protein salts; sarcosinates or acyl sarcosides such as preferably myristoyl sarcosine, TEA-lauroyl sarcosinate; as well as taurates and sodium methyl acyltaurates such as preferably sodium lauroyl taurate, sodium methyl cocoyl taurate.

Particularly suitable cationic surfactants according to the present invention encompass alkylamines such as preferably dimethyl alkylamine (dimethyl lauramine), dihydroxyethyl alkylamine dioleate, acylamidopropyldimethylamine lactate (cocamidopropyl dimethylamine lactate); alkyl imidazolines such as preferably alkyl hydroxyethyl imidazoline, Ethylhydroxymethyl oleyl oxazoline, alkyl aminoethyl imidazoline; ethoxylated alkylamines such as preferably PEG-n alkylamines, PEG-n Alkylaminopropylamine, poloxamine; quaternary compounds such as preferably tetraalkylammonium salts; alkyl trimonium chloride, PEG-n alkylmonium chloride, dialkyldimonium chloride (hydroxyethyl cetyldimonium chloride), alkylamidopropyl alkyldimonium tosylate (Cocamidopropyl ethyldimonium ethosulfate), PEG-n Acylmethyldiethonium methosulfate, dialkyl hydroxypropylmonium methosulfate, and alkyldimonium hydroxypropyl protein hydrolysate (Cocodimonium hydroxypropyl hydrolysed hair keratin).

The total amount of the at least one non-ionic surfactant in cleansing compositions according to the present invention, in particular when the composition does not comprise (i.e. is free of) soaps, is preferably selected in the range from 7.5 to 35 wt.-%, preferably from 10 to 35 wt.-%, based on the total weight of the cleansing composition. Further suitable ranges are from 7.5 to 25 wt.-%, from 10 to 25 wt.-%, from 7.5 to 20 wt.-%, from 10 to 20 wt.-%, from 15-35 wt.-%, from 20-30 wt.-% and from 22-27 wt.-%.

The total amount of the at least one zwitterionic surfactant in cleansing compositions according to the present invention, in particular when the composition does not comprise (i.e. is free of) soaps, is preferably selected in the range from 7.5 to 35 wt.-%, preferably from 10 to 35 wt.-%, based on the total weight of the cleansing composition. Further suitable ranges are from 7.5 to 25 wt.-%, from 10 to 25 wt.-%, from 7.5 to 20 wt.-%, from 10 to 20 wt.-%, from 15-35 wt.-%, from 20-30 wt.-% and from 22-27 wt.-%

It is furthermore advantageous, if the ratio (by weight) of non-ionic to zwitterionic surfactant(s) (when present in cleansing compositions according to the present invention) is selected in the range of 5:1 to 1:5, preferably 2.5:1 to 1:2.5. Even more preferably, the non-ionic surfactant(s) is used in an excess, such as in an excess of at most 50 wt.-%, more preferably at most 40 wt.-%, based on the amount of the zwitterionic surfactant.

In one particularly preferred embodiment according to the present invention, the cleansing compositions comprise at least one, preferably two to three non-ionic surfactants most preferably selected from lauryl glucoside, glyceryl stearate, PEG-100 stearate, PEG-200 hydrogenated glyceryl palmate and PEG-7 glyceryl cocoate and one zwitterionic surfactant, preferably selected from the group consisting of alkyl amidopropyl betaines, most preferably cocoamidopropyl betaine. Preferably, said cleansing compositions are free of, i.e. do not comprise soaps as defined herein.

In another particular embodiment, the cleansing compositions according to the present invention comprising at least one soap as defined herein further comprise one or more anionic, cationic, non-ionic and/or zwitterionic (also called amphoteric) surfactants with all the definitions and preferences as given above to form a surfactant mixture. Preferably the amount of said soap(s) in said surfactant mixture constitute(s) greater than 75 wt.-%, preferably greater than 80 wt.-%, more preferably greater than 85 wt.-% of the total surfactant mixture. Even more preferably in all embodiments of the present invention the surfactant mixture consists essentially of one or more soaps and one or more non-ionic surfactants. Most preferably, the surfactant mixture consists essentially of one up to four soaps and one to up to four non-ionic surfactants

Preferably said further surfactant(s) in said surfactant mixture are selected from the group consisting of anionic, non-ionic and/or zwitterionic (amphoteric) surfactants, more preferably from alkyl or alkyl ether sulfates, alkyl (poly) glucosides, (poly) glyceryl fatty acid esters polyethyleneglycol (PEG) based surfactants and alkyl amidopropyl betaines, more preferably from lauryl glucoside, glyceryl stearate, PEG-100 stearate and cocoamidopropyl betaine, most preferably from lauryl glucoside, glyceryl stearate and PEG-100 stearate, as well as any mixtures thereof. Particularly suitable in all embodiments of the present invention is a surfactant mixture consisting of two or more soaps, preferably two or three soaps and two or more non-ionic surfactants, such as in particular two to three non-ionic surfactants.

Even more preferably, the cleansing composition according to the present invention comprises either only at least one soap, preferably selected from the group consisting of sodium or potassium salts of stearic acid, palmitic, lauric acid and myristic acid as well as mixtures thereof as surfactant or a surfactant mixture, comprising at least one, preferably one to four soap(s) selected from the group consisting of sodium or potassium salts of stearic acid, lauric acid, palmitic and myristic acid as well as mixtures thereof and at least one, preferably one to three additional surfactant(s) selected from the group consisting of lauryl glucoside, glyceryl stearate and/or PEG-100 stearate.

The total amount of the further surfactant(s) in cleansing compositions according to the present invention (i.e. excluding the amount of the soap(s)) comprising the surfactant mixture is preferably selected in the range from 1 to 15 wt.-%, more preferably in the range from 1 to 10 wt.-%, most preferably in the range from 1 to 7.5 wt.-%, based on the total weight of the cleansing composition. Further suitable ranges are from 2.5 to 10 wt.-%, from 2.5 to 7.5 wt.-%, from 1 to 7.5 wt.-%, from 1 to 5 wt.-%, from 2 to 7.5 wt. % or from 2 to 5 wt.-%.

It is furthermore advantageous, if the ratio (by weight) of the total amount of the soap(s) to the total amount of additional surfactant(s) is selected in the range of 20:1 to 1:1, preferably 15:1 to 2.5:1, such as most preferably in the range of 15:1 to 3:1 or 15:1 to 5:1 or 10:1 to 3:1 or 10:1 to 5:1. Preferably, in all embodiments the soap(s) are used in an excess.

The (total) amount of all surfactants in cleansing compositions according to the present invention is preferably selected in the range of from 10 to 75 wt.-%, from 20 to 75 wt.-%, more preferably in the range from 10 to 60 wt.-% or from 25 to 60 wt.-%, most preferably in the range from 10 to 55 wt.-% or from 25 to 55 wt.-%, such as in the range of 10 to 40 wt.-% or from 30 to 40 wt.-%, based on the total weight of the cleansing composition. Further suitable ranges encompass from 10 to 30 wt.-% or from 10 to 25 wt.-%.

In all embodiments of the present invention, the cleansing compositions according to the present invention preferably comprise at least 20 wt.-% of water, more preferably at least 35 wt.-% of water, even more preferably at least 40 wt.-% of water, most preferably at least 45 wt.-% of water, such as at least 50 wt.-% of water, based on the total weight of said cleansing compositions. In particular, the water content of the cleansing compositions in all embodiments of the present invention is selected in the range from 20 to 75 w.-%, most preferably in the range from 20 to 65 wt-%, based on the total weight of the cleansing composition. Further suitable ranges encompass 25 to 75 wt. %, 25 to 60 wt.-%, 25 to 55 wt.-%, 30 to 75 wt. %, 30 to 70 wt.-%, 30 to 60 wt.-%, to 75 wt. %, 40 to 65 wt.-% and 40 to 60 wt.-%.

The cleansing compositions of the present invention may also include one or more optional ingredients such as a pearlescent or opacifying agent, a thickening agent, humectants, chelating agents, and additives which enhance their appearance, feel and fragrance, such as colorants, fragrances, preservatives, pH adjusting agents, and the like.

Commercially available pearlescent or opacifying agents which are capable of suspending water insoluble additives and/or which tend to indicate to consumers that the resultant product is a cleansing composition are suitable for use in this invention. The pearlescent or opacifying agent may be present in an amount, based upon the total weight of the composition, of from about 1 to 10 wt.-%, preferably from about 1.5 to 7 wt.-%, and more preferably, from about 2 to 5 wt.-%.

Examples of suitable pearlescent or opacifying agents include, but are not limited to mono or diesters of (a) fatty acids having from about 16 to about 22 carbon atoms and (b) either ethylene or propylene glycol mono or diesters of (a) fatty acids having from about 16 to about 22 carbon atoms, (b) a polyalkylene glycol of the formula: HO-(JO)a-H, wherein J is an alkylene group having from about 2 to about 3 carbon atoms and a is 2 or 3; fatty alcohols containing from about 16 to about 22 carbon atoms; fatty esters of the formula: KCOOCH₂L, wherein K and L independently contain from about 15 to about 21 carbon atoms; inorganic solids insoluble in the cleansing composition, and mixtures thereof.

The pearlescent or opacifying agent may be introduced to the cleansing composition as a pre-formed, stabilized aqueous dispersion, such as that commercially available from Henkel Corporation of Hoboken, New Jersey under the tradename, “Euperlan PK-3000.” This material is a combination of glycol distearate (the diester of ethylene glycol and stearic acid), Laureth-4 (CH₃(CH₂)₁₀CH₂ (OCH₂CH₂)₄OH) and cocamidopropyl betaine and preferably is in a weight percent ratio of from about 25 to about 30:about 3 to about 15:about 20 to about 25, respectively.

Commercially available thickening agents, which are capable of imparting the appropriate viscosity to the cleansing compositions are suitable for use in this invention. If used, the thickener should be present in the compositions in an amount sufficient to raise the Brookfield viscosity of the composition to a value of between about 500 to about 10,000 centipoise. Examples of suitable thickening agents nonexclusively include: mono or diesters of 1) polyethylene glycol of formula: HO—(CH₂CH₂O)_{z}H, wherein z is an integer from about 3 to about 200; and 2) fatty acids containing from about 16 to about 22 carbon atoms; fatty acid esters of ethoxylated polyols; ethoxylated derivatives of mono and diesters of fatty acids and glycerine; hydroxyalkyl cellulose; alkyl cellulose; hydroxyalkyl alkyl cellulose; and mixtures thereof. Preferred thickeners include polyethylene glycol ester, and more preferably PEG-150 distearate which is available from the Stepan Company of Northfield, Illinois or from Comiel, S.p.A. of Bologna, Italy under the tradename, “PEG 6000 DS”. The amount of thickener(s) in the cleansing composition is preferably selected in the range from 0 to 7 wt.-%, preferably from 1 to 5 wt.-%, most preferably from 2 to 4 wt.-%, based on the total weight of the cleansing composition. Further suitable ranges encompass 0 to 2.5 wt.-% and 0 to 1 wt.-% and 1 to 3 wt.-%.

The cleansing compositions according to the present invention advantageously further comprise one or more polyethylene glycols (PEGs) which can be used as binders, humectants and/or solvents. A particularly preferred polyethyleneglycol for the use in the cleansing compositions is PEG-32. When present, the amount of said PEG(s) in the cleansing composition according to the present invention is preferably selected in the range from 0.1 to 10 wt.-%, preferably from 1 to 7.5 wt.-%, most preferably from 2.5 to 7.5 wt.-%, based on the total weight of the cleansing composition. Further suitable ranges encompass 0 to 7.5 wt.-% and 1 to 7.5 wt.-% and 4 to 7.5 wt.-%.

Commercially available humectants, which are capable of providing moisturization and conditioning properties to the cleansing composition, are suitable for use in the present invention. Examples of suitable humectants nonexclusively include: 1) water soluble liquid polyols such as glycerol; 2) polyalkylene glycol of the formula: HO—(R″O)b-H, wherein R″ is an alkylene group having from about 2 to about 3 carbon atoms and b is an integer of from about 2 to about 10; 3) polyethylene glycol ether of methyl glucose of formula CH₃—C₆H₁₀O₅—(OCH₂CH₂)c-OH, wherein c is an integer from about 5 to about 25; 4) urea; 5) hyaluronic acid or derivatives thereof such as sodium hyaluronate and 6) mixtures thereof, with glycerol being the preferred humectant.

Preferably, in all embodiments of the present invention, the cleansing composition further comprise at least one humectant. The amount of the at least on humectant such as preferably glycerol and/or sodium hyaluronate is preferably selected in the range from 0 to 90 wt.-%, preferably from 2.5 to 40 wt.-%, most preferably from 4 to 10 wt.-%, based on the total weight of the cleansing composition. Further suitable ranges encompass 0.01 to 7.5 wt.-%, 1 to 7.5 wt.-%, 1 to 5 wt.-%, 2.5 to 7.5 wt.-% and 2.5 to 5 wt.-%.

Examples of suitable chelating agents include those which are capable of protecting and preserving the compositions of this invention. Preferably, the chelating agent is ethylenediamine tetraacetic acid (‘EDTA’), and more preferably is tetrasodium EDTA, available commercially from Dow Chemical Company of Midland, Michigan under the tradename, “Versene 100XL” or even disodium EDTA, commercially available from BASF under tradename “EDETA BD” and is present in an amount, based upon the total weight of the composition, from about 0 to 0.5 wt.-%, and preferably from about 0.01 to 0.25 wt.-%, more preferably from 0.025 to 0.25 wt.-%, such as most preferably from 0.05 to 0.25 wt.-%. Further suitable ranges encompass from about 0.01 to 0.2 wt.-%, more preferably from 0.025 to 0.2 wt.-%, such as most preferably from 0.05 to 0.15 wt.-%.

In a still further advantageous aspect of the invention, the cleansing compositions of the present invention further comprise a preservative and/or a preservative booster, such as e.g. quaternium-15. When present, the preservative respectively the preservative booster is preferably used in an amount of 0.01 to 2 wt. %, more preferably in an amount of 0.05 to 1.5 wt.-%, most preferably in an amount of 0.1 to 1.0 wt.-%, based on the total weight of the cleansing composition.

The cleansing compositions according to the present invention may further comprise a skin and/or hair conditioning and/or soothing actives such as e.g. allantoin, d- or dl-panthenol, ethyl panthenol, bisabolol, phytantriol, vitamins, plant extracts and hyaluronic acid or salts thereof such as sodium hyaluronate. The amount of such skin actives, when present, is preferably selected in the range from 0.01 to 1 wt.-%, more preferably from 0.025 to 0.5 wt. %, most preferably from 0.05 to 0.25 wt.-%, based on the total weight of the cleansing composition.

The pH of the cleansing compositions of this invention is preferably maintained in the range from about 4.5 to about 10.5, and more preferably from about 5.0 to about 10.0.

The compositions of the present invention may furthermore be “substantially free” of oils or silicones. As used herein, “substantially free” shall mean that the cleansing composition contains, based upon the total weight of the composition, less than about 1 wt.-%, for example, less than about 0.5 wt.-% or less than about 0.2 wt.-% oils and/or silicones.

Particularly suitable cleansing compositions according to the present invention are rinse-off compositions, even more in particular liquid aqueous rinse-off compositions.

The term ‘rinse-off’ as used herein is defined as per the Regulation (EC) No. 1223/2009 of the European Parliament and of the Council of 30 Nov. 2009 on cosmetic products (recast), i.e. a cosmetic product or composition is a rinse-off one when it is intended to be removed after application on skin, hair or mucous membranes of a human subject. The rinse-off cosmetic composition presently disclosed may be a rinse-off, cosmetic cleansing composition, for example a rinse-off, cosmetic, personal care, cleansing composition.

Advantageously, the term ‘liquid’ as used herein means that the composition has a viscosity lower than about 65000 cPs, such as lower than about 60000 and greater than for example about 500 cPs, or greater than about 1000 cPs or greater than about 2000 cPs, or greater than 5000 cPs or greater than 10000 cPs when measured at 20-25° C. using a Brookfield viscometer at 10 rpm. The choice of an appropriate spindle can be made by a skilled man based on e.g. information provided by viscometer manufacturers and the expected viscosity range of the composition to be tested.

Examples of the rinse-off cosmetic composition according to the present invention are shower gels (body shampoos), liquid soaps, wash gels, body washes, soap bars, foam baths, (hair) shampoos as well as shaving preparations. Said compositions are not edible or comestible, like toothpastes. Particularly preferred rinse-off compositions according to the present invention are liquid aqueous rinse off compositions such as shower gels (body shampoos), liquid soaps, wash gels, body washes foam baths and (hair) shampoos.

In a particular advantageous aspect, the cleansing compositions according to the present invention are free of any parabenes, benzethoniumchlorid, piroctone olamine, lauroylarginat, methylisothiazolinon, chlormethylisothiazolinon, bronopol, benzalkoniumchloride, formaldehyd releasing compounds, salicylic acid, triclosan, DMDM hydantoin, chlorphenesin and IPBC (lodopropinylbutyl carbamate), such as in particular free of methylchloroisothiazolinone.

In a further embodiment the cleansing compositions according to the present invention are sulfate-free. The term ‘sulfate-free’ as used herein refers to cleansing compositions which do not comprise sulfates, i.e. do not comprise any organic compounds that include a sulfate moiety (—OSO₃²⁻) such as e.g. laureth sulfates or lauryl sulfates; a composition may however include sulfonates, and still be considered “sulfate-free” or “free of sulfates” as those terms are used herein. It is well understood that in particular no sulfate-based surfactants i.e. surfactants comprising sulfate groups such as sodium or ammonium lauryl sulfate or sodium or ammonium laureth sulfate are present in the cleansing compositions according to the present invention.

In a further particular embodiment, the invention also relates to a method for washing the skin and/or hair, said method comprising applying a cleansing composition according to the present invention with all the definitions and preferences as given herein to the skin and/or hair, followed by washing (rubbing and/or distributing the composition onto the skin to produce foaming) and then rinsing the composition off the skin again.

In another embodiment, the present invention also relates to the use of a cleansing composition according to the present invention with all the definitions and preferences as given herein for washing the skin and/or the hair.

The cleansing composition of the present invention may be used on the body in conjunction with any personal cleansing implement known in the art such as a washcloth, a mesh or apertured film, pouf, sponge, brush and the like. The composition may be marketed together with one or more of such implements in a kit.

The following examples are provided to further illustrate the compositions and effects of the present invention. These examples are illustrative only and are not intended to limit the scope of the invention in any way.

### EXAMPLES

### Procedure

Add 2.5 g of a 1% solution of the respective cleanser in a tube (Diameter 2.2 cm, height 15 cm)
Add 2.5 g of water (reference) or a 1% aqueous solution of the respective HMO
Note the height of liquid in the tube
Shake 15 s (close the test tube with a stopper)
Note the max height of the mixture (based on the foam produced)
Result is the difference between the maximum height of the reference versus the test sample after shaking.

### HMO's Abbreviation

2′-FL: 2′-Fucosyllactose
3-FL: 3-Fucosyllactose
LNnT: Lacto-N-neotetraose
LNT: Lacto-N-tetraose
FL/DFL: 2′-Fucosyllactose/Difucosyllactose mixture (75% 2′-FL; >5% DFL)

### 1. Foaming Properties of HMO's

### 1.1. In the Presence of a Soap

TABLE 1.1.a
Formulation ‘Cream cleanser’

| Ingredients | INCI Name | wt-% |
|---|---|---|
| Water Dem | AQUA | ad 100 |
| Edeta BD | DISODIUM EDTA | 0.05 |
| Humectant | PEG-32 (POLYGLYKOL 1500 S) | 5 |
| Soap | LAURIC ACID | 3.3 |
| | STEARIC ACID | 12 |
| | MYRISTIC ACID | 11.5 |
| Non-ionic surfactant | GLYCERYL STEARATE; PEG-100 | 2 |
| | STEARATE (Tego Care 165) | |
| | LAURYL GLUCOSIDE | 4 |
| | (Plantacare 1200 UP) | |
| Potassium Hydroxide | POTASSIUM HYDROXIDE | 4.65 |

TABLE 1.1.b
Results: Foam volume using 1% HMO solution

| # | HMO | Foam volume [mm] | %* |
|---|---|---|---|
| Reference (control) | — | 2 | 100 |
| Inv-1 | 2′-FL | 4 | 200 |
| Inv-2 | 3-FL | 3 | 150 |
| Inv-3 | LNnT | 4 | 200 |
| Inv-4 | LNT | 8 | 400 |
| Inv-5 | 2′-FL/DFL | 6 | 300 |

*based on reference set at 100%

The samples according to present invention showed a significantly higher foam volume highlighting the excellent foam boosting properties of the respective HMO.

The same experiment has been repeated using 0.5% and 2% HMO solutions, respectively 0.5% and 2% maltose solutions (reference). The results are depicted in table 1.1.c below.

TABLE 1.1.c
Results: Foam volume using 0.5% and 2% HMO solutions
respectively 0.5% and 2% maltose solutions

| # | HMO | 0.5%* | 2%* |
|---|---|---|---|
| Reference (control) | — | 100% | 100% |
| Reference maltose | — | 133% | 100% |
| Inv-6 | 2′-FL | 233% | 200% |
| Inv-7 | 3-FL | 200% | 350% |
| Inv-8 | LNnT | 167% | 150% |
| Inv-9 | LNT | 200% | 400% |
| Inv-10 | 2′-FL/DFL | 167% | 200% |

*based on reference set at 100%

As can be retrieved from the results, also lower as well as higher concentrations of the respective HMO's led to an increase in the foam volume, while maltose does not improve the foaming properties, respectively not to the same extend.

### 1.2. In the Presence of Other Surfactants

TABLE 1.2.a
Formulation sensitive skin cleanser

| Ingredients | wt-% |
|---|---|
| Water Dem | ad 100 |
| Allantoin | 0.1 |
| Chelating agent (Edeta ® BD) | 0.05 |
| Hyaluraonic acid | 0.01 |
| Diols | 6 |
| Preservative(s) | 0.1 |
| Zwitterionic surfactant (cocamidopropyl betaine) | 25 |
| Non-ionic surfactant (lauryl glucoside; | 25 |
| PEG-200 hydrogenated glyceryl palmate; | |
| PEG-7 glyceryl cocoate) | |
| Citric Acid | 0.3 |

TABLE 1.2.b
Results: Foam volume

| # | HMO | Foam volume [mm] | %* |
|---|---|---|---|
| Reference | — | 19 | 100 |
| Inv-11 | 2′-FL | 20 | 105 |
| Inv-12 | 3-FL | 29 | 153 |
| Inv-13 | LNnT | 26 | 137 |
| Inv-14 | LNT | 25 | 132 |
| Inv-15 | 2′-FL/DFL | 25 | 132 |

*based on reference set at 100%

Compared to the reference, also here the samples showed an improved foaming highlighting the excellent foam boosting properties of the respective HMO, which are however less pronounced compared to the formulation comprising at least one soap.

The same experiment has been repeated using 2% HMO solutions, respectively a 2% maltose solutions (reference). The results are depicted in table 2.1.c below.

TABLE 2.1.c
Results: Foam volume using 2% HMO solution
respectively 2% maltose solutions

| # | HMO | 2%* |
|---|---|---|
| Reference (control) | — | 100 |
| Reference maltose | — | 117% |
| Inv-16 | LNnT | 121% |
| Inv-17 | LNT | 133% |

*based on reference set at 100%

Compared to the reference, also these samples showed an improved foaming highlighting the foam boosting properties of the respective HMO over a wide range of cleansing compositions. The use of maltose did not result in equally advantageous results.

1. A cleansing composition comprising at least one soap selected from the group consisting of sodium or potassium salts of aliphatic acids having from about 8 to 22 carbon atoms and one or more human milk oligosaccharide selected from the group consisting of 2′-fucosyllactose, 3-fucosyllactose, difucosyllactose, lacto-N-fucopentaose I, lacto-N-neotetraose and lacto-N-tetraose as well as mixtures thereof.

2. The cleansing composition according to claim 1, wherein the amount of the at least one human milk oligosaccharide is selected in the range from 0.01 to 10 wt.-%, preferably in the range from 0.1 to 7.5 wt.-%, most preferably in the range from 0.2 to 5 wt.-%, based on the total weight of the composition.

3. The cleansing composition according to claim 1, wherein the at least one human milk oligosaccharide is selected from the group consisting of 2′-fucosyllactose, difucosyllactose, lacto-N-neotetraose and lacto-N-tetraose as well as mixtures thereof, preferably from lacto-N-tetraose and a mixture of difucosyllactose and 2′-fucosyllactose.

4. The cleansing composition according to claim 1, wherein the at least one soap is selected from the group consisting of sodium or potassium salts of aliphatic acids having from about 8 to about 20 carbon atoms, most preferably from about 10 to about 18 carbon atoms as well as mixtures thereof.

5. The cleansing composition according to claim 1, wherein the at least one soap is selected from the group consisting of sodium or potassium salts of stearic acid, lauric acid and myristic acid as well as mixtures thereof.

6. The cleansing composition according to claim 1, wherein the amount of the at least one soap is selected in the range from 3 to 95 wt.-%, based on the total weight of the composition.

7. The cleansing composition according to claim 1, wherein the composition comprises at least one additional surfactant selected from the group consisting of consisting of anionic, non-ionic and/or zwitterionic (amphoteric) surfactants, preferably the composition comprises at least one additional non-ionic surfactant.

8. The cleansing composition according to claim 7, wherein the at least one additional surfactant is selected from the group consisting of alkyl or alkyl ether sulfates, alkyl (poly)glucosides, (poly)glyceryl fatty acid esters, polyethyleneglycol (PEG) based surfactants and alkyl amidopropyl betaines, preferably from lauryl glucoside, glyceryl stearate, PEG-100 stearate and cocoamidopropyl betaine as well as any mixtures thereof, most preferably the composition further comprises lauryl glucoside, glyceryl stearate and PEG-100 stearate.

9. The cleansing composition according to claim 1, wherein the cleansing composition comprises at least 35 wt.-% of water, preferably at least 40 wt.-% of water, most preferably at least 45 wt.-% of water, based on the total weight of the cleansing composition.

10. The cleansing composition according to claim 1, wherein the cleansing composition is a rinse-off composition.

11. The cleansing composition according to claim 10, wherein the cleansing composition is a shower gel, a liquid soap, a wash gel, a body wash, a soap bar, a foam bath, a shampoo or a shaving preparation.

12. The cleansing composition according to claim 1, wherein the cleansing composition is a liquid soap, a cream soap, a melt or a pour soap bar and the amount of the at least one soap selected in the range from 3 to 35 wt.-%, more preferably in the range from 5 to 30 wt.-%, most preferably in the range from 7 to 20 wt.-%, based on the total weight of the cleansing composition.

13. Use of at least one human milk oligosaccharide as foam enhancing agent or foam booster, preferably to enhance foam volume.

14. The use according to claim 13, wherein the at least one human milk oligosaccharide is selected from the group consisting of 2′ fucosyllactose, 3-fucosyllactose, difucosyllactose, lacto-N-fucopentaose I, 3′sialyllactose sodium salt, 6′sialyllactose sodium salt, lacto-N-neotetraose and tacto-N-tetraose as well as mixtures thereof, preferably from 2′-fucosyllactose, difucosyllactose and lacto-N-neotetraose and lacto-N-tetraose as well as mixtures thereof, most preferably from lacto-N-tetraose and a mixture of difucosyllactose and lacto-N-fucopentaose.

15. A method of increasing the foaming properties of cleansing compositions comprising at least one surfactant, preferably at least one soap, said method comprising the step of adding at least one human milk oligosaccharide into said cleansing composition.
